(19) United States
(12) Patent Application Publication
(10) Pub. No.: US 2008/0114336 A1
(43) Pub. Date: May 15, 2008

(54) INTESTINAL TRACT CLEANSING APPARATUS

(76) Inventor: Tomio SEI, Tokyo (JP)

Correspondence Address: MCGLEW AND TUTTLE, P.C., SCARBOROUGH STATION, SCARBOROUGH, NY, 10510-9227, US

(21) Appl. No.: 11/876,962

(22) Filed: Oct. 23, 2007

(30) Foreign Application Priority Data
Nov. 9, 2006 (JP) 2006-303586

(51) Int. Cl. A61M 1/00

(52) U.S. Cl. 604/540

(57) ABSTRACT

A filter apparatus, namely the first filter apparatus, the second filter apparatus, and the third filter apparatus, filters water from the faucet introduced into the intestinal tract cleansing apparatus. A heating unit heats the filtered water. The reverse osmosis membrane apparatus generates pure water by utilizing reverse osmosis membrane to treat the heated water. A high-pressure regulation unit pressurizes the pure water treated with the reverse osmosis membrane, and floods the pure water as pure water for cleansing an intestinal tract of human beings from a flooding opening.

### BACKGROUND OF THE INVENTION

1. Field of the Invention

The present invention relates to an intestinal tract cleansing apparatus, and more particularly to an intestinal cleansing apparatus for removing impurities such as bacteria contained in raw water, and obtaining water for intestinal cleansing with a value below a specified impurity concentration, and by using the water obtained, this intestinal tract cleansing apparatus is to cleanse the intestine tract.

2. Description of the Related Art

Formerly, as an intestinal tract cleansing apparatus, for example, the lower intestinal tract cleansing apparatus was proposed, which forms the cleansing water by filtering and heating, ozonizes said cleansing water, and then introduces the ozonized cleansing water with a hose into the lower intestinal tract of a patient (Japanese patent application laid open No. 2004-024846).

If the cleansing water is ozonized, it means introducing the ozonized cleansing water into the intestinal tract of a human being. However, ozone being carcinogenic, introducing the ozonized cleansing water into the intestinal tract may have an adverse effect on human health, and is not desirable.

Moreover, even if ozone sterilizes bacteria contained in the cleansing water, bacterial residues (bacterial residual substances) still remain in the cleansing water. For this reason, it is necessary not only to sterilize bacteria contained in the cleansing water, but also to remove bacteria themselves.

On the other hand, concerning potable water, the criterion of its impurity concentration is stipulated. For example, in Japan, it is 60 ppm, and in the United States of America, it is 40 ppm. The cleansing water to use for cleansing intestinal tract differs from potable water, however, there is no difference in the point that it is introduced into a human body. Accordingly, it is desirable that the impurity concentration of the cleansing water (ppm) should satisfy the impurity concentration's criterion of potable water.

### SUMMARY OF THE INVENTION

It is an object of the present invention to provide an intestinal tract cleansing apparatus for removing impurities such as bacteria contained in raw water, and obtaining the water of which value is below specified impurity concentration to use for intestinal tract cleansing.

An intestinal tract cleansing apparatus of the present invention flushes output water for cleansing an intestinal tract from a flooding opening, and includes a filter apparatus filtering water, a heating unit heating the water, and a reverse osmosis membrane unit treating the water with reverse osmosis. Wherein the water filtered with the filter apparatus is heated with the heating unit, and treated with reverse osmosis with the reverse osmosis membrane unit to output the water for cleansing the intestinal tract from the flooding opening.

Preferably, the intestinal tract cleansing apparatus of the present invention further includes a pressure regulation unit adjusting water pressure at the flooding opening.

Preferably, the intestinal tract cleansing apparatus of the present invention further includes a switching unit switching direction of the output of the water for cleansing the intestinal tract between the flooding opening and drainage opening provided separately with the flooding opening.

The intestinal tract cleansing apparatus of the present invention filters the raw water (for example, the water from the faucet), treats this filtered water with heat and reverse osmosis to obtain water below (referred to as pure water hereinafter) the stipulated impurity concentration value (for example, 10 ppm), and flushes out this water from flooding opening as the intestinal cleansing water. For example, the intestinal tract cleansing apparatus of the present invention obtains the pure water of which impurity concentration is 10 ppm, through the filtration of water from a faucet in Tokyo, of which impurity concentration is 120 ppm. This is to be heated up to body temperature (for example, 37), and flushed out (for example, 40 cc/min-100 cc/min) from the flooding opening at a suitable pressure (for example 3.0 Kg/cm²) and quantity in order to cleanse the intestinal tract. Consequently, concerning the impurity concentration of the water for cleansing the intestinal tract, it is possible to cleanse the intestinal tract with purified water that, for example, satisfies the criterion of potable water (for example, it is 60 ppm for Japan and 40 ppm for the United States of America). In addition, it is possible to obtain pure water for cleansing that is not carcinogenic, since the water introduced into the intestinal tract cleansing apparatus is not ozonized. Moreover, it is possible to obtain the pure water for cleansing that does not contain bacteria (and its residue) since it is possible to remove bacterial residual substance by the means of reverse osmosis treatment from the water introduced in the intestinal tract cleansing apparatus.

Furthermore, with the intestinal tract cleansing apparatus of the present invention, it is possible to regulate the water pressure in the flooding opening. Accordingly, it is possible to regulate the pressure of the flooding pure water for cleansing to the extent that, for example, it will not damage the intestinal tract of human beings.

In addition, with the intestinal tract cleansing apparatus of the present invention, the direction of the pure water for cleansing can be changed between the flooding opening and the other flooding opening set up separately. This helps to clean the inside per se of the intestinal tract cleansing apparatus by flushing out the water from the drainage opening for a specified time. As a result, it is possible to obtain pure water for cleansing with lower impurity concentration.

### BRIEF DESCRIPTION OF THE DRAWINGS

FIG. 1 shows an illustration of the external appearance of the intestinal tract cleansing apparatus of the present invention.

FIG. 2 shows an illustration of the configuration of the intestinal tract cleansing apparatus of the present invention.

FIG. 3 shows an illustration of the external appearance of the flooding nozzle.

FIG. 4 shows an illustration of another configuration of the intestinal tract cleansing apparatus of the present invention.

### DETAILED DESCRIPTION OF THE PREFERRED EMBODIMENTS

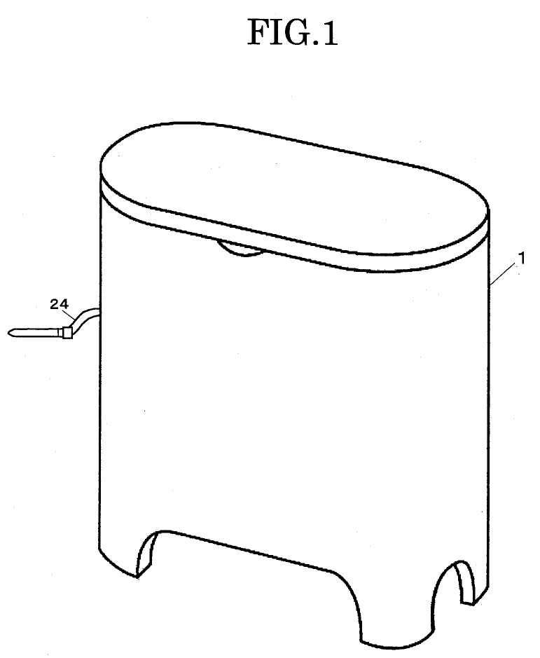

FIG. 1 shows an illustration of the external appearance of the intestinal tract cleansing apparatus of the present invention. The intestinal tract cleansing apparatus is equipped with a cabinet (or body) 1 in the form shown in FIG. 1. The cabinet 1 of the intestinal tract cleansing apparatus may be installed, for example, on the side of the toilet water storage tank.

Concerning the raw water introduced in the cabinet 1, the intestinal tract cleansing apparatus carries out the treatment (hereinafter called reverse osmosis treatment) utilizing the reverse osmosis membrane (reverse osmosis effect) and the heat treatment after the filtration treatment. Consequently, the intestinal tract cleansing apparatus obtains the water (pure water) for cleansing the intestinal tract with a suitable water pressure at an appropriate temperature of which impurity concentration is exceedingly low. This water is to be introduced via the flooding nozzle 24 into the intestinal tract of human beings (or animals) to cleanse the intestinal tract. For raw water, water from a faucet, for example, may be used (or the water purified to a certain extent by other purifying apparatus).

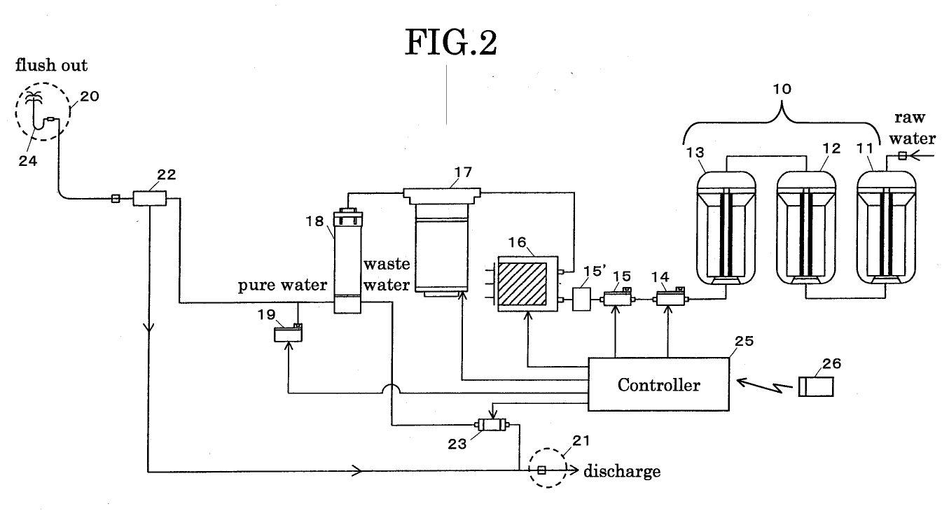

FIG. 2 is an illustration of the configuration of the intestinal tract cleansing apparatus of the present invention. The intestinal tract cleansing apparatus is equipped with a running water system for flushing out the pure water for cleansing obtained from the raw water, and a control system for controlling the treatment in the running water system.

The running water system is equipped with a filter 10, a solenoid valve (electromagnetic valve) 14, a low-pressure regulation unit 15, an ultraviolet sterilizer 16′, a heating unit 16, a pressure pump 17, a reverse osmosis membrane apparatus 18, a high-pressure regulation unit 19, a flooding opening 20, a drainage opening 21, a switch 22, a drainage valve 23, and a flooding nozzle 24. The filter 10 compromises the first filter 11, the second filter 12, and the third filter 13.

The first filter apparatus 11 is connected with the feed opening of the raw water (the water entrance or introductory opening into the cabinet 1). For example, if using the water from faucet as the raw water, it would be connected to the faucet through, for example, a valve (or without it). The first filter apparatus 11 is equipped with a filter to purify the raw water introduced into the intestinal tract cleansing apparatus. The first filter apparatus 11 has, for example, the perforations of diameter, and using this, it can roughly filter the impurities contained in the raw water, prevent the clogging of the pore of the active carbon of the second filter apparatus 12, and prevent the clogging of the filter's perforations of the third filter apparatus 13.

The second filter apparatus 12 is equipped with a filter (active carbon filter) to adsorb, chlorine, microorganism, organic matters and suchlike through the active carbon. The active carbon of the second filter apparatus 12 adsorbs chlorine, microorganism, organic matters and suchlike in this pore. These are the impurity, which are not purified at the first filter apparatus 11. Using this, it is possible to reduce the impurity concentration (ppm) of the water roughly filtered at the first filter apparatus 11, and at the same time, prevent the clogging of the third filter apparatus 13.

The third filter apparatus 13 is equipped with a filter to further filter the water filtered at the second filter apparatus 12. The filter of the third filter apparatus 13 has smaller perforations than the first filter apparatus 1, for example, the perforations of 1 μm in diameter. The filter treatment conducted at the third filter apparatus 13, makes it possible to prevent the clogging at the reverse osmosis membrane apparatus 18 provided at the back (the flooding opening 20 side) of third filter 13 apparatus within the concerned running water system.

By configuring the filter apparatus 10 with the separate filters of the first filter apparatus 11, the second filter apparatus 12, and the third filter apparatus 13, it is possible to replace them individually. Accordingly, even when the filtering performance of the filter apparatus 11 declines, for example, it should be sufficient to replace only the concerned filter apparatus 11, and there should be no need for replacing all the filters of the filter apparatus 10.

In addition, it is possible to filter without clogging (in an efficient fashion) by providing the two individual filters of the same kind as the first filter apparatus 11 and the third filter apparatus 13, rather than utilizing a single large filter. Moreover, providing the heterogeneous second filter apparatus 12 between the first filter apparatus 11 and the third apparatus 13 which are the same kind, it is possible to roughly filter the relatively large and diverse impurities through the first filter apparatus 11 and the second filter apparatus 12, and to filter without clogging (in an efficient fashion) through the third filter apparatus 13.

The solenoid valve 14 controls the water flow from the low-pressure regulation unit 15 by switching “ON” and “OFF”. Namely, when the solenoid valve 14 is “OFF”, water does not flow to the low-pressure regulation unit 15, regardless of the condition of the faucet (and the valve). Accordingly, it is possible to leave the first filter apparatus 11 connected to the water faucet. Water flows towards the low-pressure regulation unit 15 when the solenoid valve is switched “ON”. Switching “ON” or “OFF” of the solenoid valve is controlled with the controller 25.

The low-pressure regulation unit 15 is the water pressure sensor and concurrent pressure regulation valve. The low-pressure regulation unit 15 detects the water pressure, and adjusts the water pressure to a suitable value, which allows the operation of the first filter apparatus 11 to the heating unit 16. It increases the water pressure from the faucet when it is too low, and lowers the water pressure when it is too high. By doing this, it adsorbs the difference between the water pressure of the faucet and the water pressure at the pressure pump 17 of the running water system. The water pressure regulation by the low-pressure regulation unit 15 is controlled by the controller 25.

Ultraviolet sterilizer 16′ irradiates the water introduced into the ultraviolet sterilizer 16′ with an ultraviolet beam to get rid of the bacteria in the water. Temperature increase accompanies with the bacteria treatment through the ultraviolet sterilizer 16′. Accordingly, It is desirable to install the ultraviolet sterilizer 16′ at the front of the heating unit 16 (on the water conduit side), as to allow the temperature increase to be adsorbed at the heat unit 16 described below. In this example, the ultraviolet sterilizer 16′ is to be installed in the heat unit 16.

Moreover, the controller 25, described below, controls the heat treatment, which takes into consideration the temperature increase in the ultraviolet sterilizer 16′ in the heating unit 16, when installing the ultraviolet sterilizer 16′ at the back of (at the side of the flooding opening 20) of the heating unit 16.

The heating unit 16 heats the water introduced in the heating unit 16 (the water filtered with the filter apparatus 10). The heating unit 16 is equipped with a tank and a heater (both are not illustrated), and heats the water stored in the tank with the heater. By doing this, it regulates the water temperature flushing out of the flooding opening 20 to temperature suitable for introducing the water into the intestinal tract. This temperature should be about 37, for example. Namely, the temperature should be close to the body temperature, but slightly higher than that. As a result of this, the heated water could prevent inflicting scalds to the patient's intestines, and causing the patient being unwell (or causing discomfort to the patient), such as hypothermia and suchlike. The heating unit 16 is controlled by the controller 25.

The pressure pump 17 adds the pressure necessary for reverse osmosis processing with the reverse osmosis membrane apparatus 18 for the water introduced from the heating unit 16. Namely, it adds the pressure higher than the osmotic pressure of the water molecule to the water introduced from the heating unit 16 (from the side of the water with high impurity concentration). The pressure treatment through the pressure pump 17 is controlled by the controller 25.

The reverse osmosis membrane apparatus 18 is equipped with a reverse osmosis membrane, and carries out the reverse osmosis treatment of the water introduced from heating unit 16 to separate them into pure water and wastewater. The reverse osmosis membrane of the reverse osmosis membrane apparatus 18 permeates only the pure water, but the impurities, such as the ion electrolyte dissolved in the water filtered through the filter apparatus 10. By doing this, it is possible to remove the impurities, such as viruses, bacteria, and chemicals substance such as trihalomethane. The pure water for cleansing of less than 10 ppm, for example, can be obtained with the reverse osmosis treatment through the reverse osmosis membrane apparatus 18.

The reverse osmosis membrane apparatus 18 and the pressure pump 17 constitute the reverse osmosis membrane unit, which carries out reverse osmosis treatment of the water introduced. However, the reverse osmosis membrane apparatus 18 can be replaced on its own, independently from the pressure pump 17. By doing this, whenever the performance of reverse osmosis membrane apparatus 18 declines, it should be sufficient to replace only the concerned reverse osmosis membrane apparatus 18, and there should be no needs for replacing the pressure pump 17. In addition, by providing separately the filter apparatus 10 and the reverse osmosis membrane apparatus 18, it is possible to replace only either one of these at each time.

The high-pressure regulation unit 19 is the water pressure sensor and concurrent pressure regulation valve. Though not illustrated, it temporarily takes in the pure water for cleansing from the reverse osmosis membrane apparatus 18, and then flushes them out. At this time, the high-pressure regulation unit 19, sensors the water pressure of the pure water for cleansing, and regulates the water quantity and water pressure of the floodwater at the flooding opening 20. Namely, this pressure is controlled at 1 Kg/cm²-3 Kg/cm², for example, as to flush the pure water for cleansing comfortably into the interior bit of the intestinal tract. At this time, the upper limit of the water pressure is to be regulated to 3 Kg/cm², for example, so that the pure water for cleansing does not injure the intestinal tract of the patient. In addition, the quantity of the flooding pure water for cleansing is to be regulated to 40 cc/min-100 cc/min, for example, as not to damage the health of the patient (or causing discomfort to the patient), as a result of sudden (or excessive) distention of intestinal tract. The regulation of quantity and pressure of flooding water through the high-pressure regulation unit 19 may be controlled from the controller 25.

The flooding opening 20 is the exit of the pure water for cleansing. The impurity concentration of the pure water for cleansing flushed out of the flooding opening is 1 ppm-15 ppm, for example. This level satisfies the potable water criterion (for example, it is 60 ppm for Japan, and 40 ppm for the United States of America). Consequently, it possible to clean the water introduced to the intestinal (internal) tract even purer than the potable water. The flooding nozzle 24 is to be installed on the flooding opening 20.

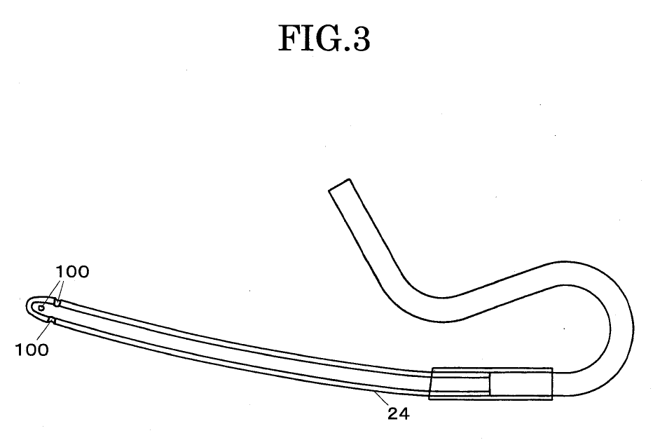

The flooding nozzle 24 is the attachment of the flooding opening 20. The pure water for cleansing is introduced into the intestinal tract through this duct. An illustration of the exterior appearance of the flooding nozzle 24 is shown in FIG. 3. The complete shape of the flooding nozzle 24 in FIG. 3 is like the deformed shape of the letter N. The extremity of the flooding nozzle 24 is round to make the shape of the flooding nozzle 24 suitable to insert into the intestinal tract. In addition, the surface of the extremity of the flooding nozzle 24 is provided with alternate (zigzag fashion) several hundred flooding pores. With this, it is possible to flood evenly the pure water for cleansing from the concerned several hundred flooding pores into the intestinal tract.

The drainage opening 21 is provided separately from the flooding opening 20. Principally, it is the exit of the wastewater separated by reverse osmosis treatment with the reverse osmosis membrane apparatus 18. The impurity concentration of the wastewater ejected from the drainage opening 21 is 180 ppm-200 ppm, for example.

The switch 22 is the switching unit for the flooding of the pure water for cleansing between the flooding opening 20 and the drainage opening 21. The user of the intestinal tract cleansing apparatus, for example, can manually switch on the switch 22. Moreover, it is possible to make the controller 25 to switch on the switch 22, according to the instruction given from the remote controller 26 described below.

When washing the inside of the running water system (at the time of cleansing operation), the switch 22 switches the flooding extremity of the pure water for cleansing from the reverse osmosis membrane apparatus 18 to the drainage opening 21. When using the pure water for cleansing (at the time of cleansing the intestinal tract), the switch 22 switches the flooding extremity of the pure water for cleansing from the reverse osmosis membrane apparatus 18 to the flooding opening 20. The impurity concentration of the pure water for cleansing flooding from the flooding opening 20 is about 20 ppm, when not cleaning the inside of running water system. This is because the impurities, such as microorganism, increase in either side of the internal running water system. However, the impurity concentration of the pure water for cleansing flooding from the flooding opening 20 becomes approximately 10 ppm-1 ppm, when conducting washing operations for 15-30 minutes almost everyday.

The drainage valve 23 controls the ejection from the drainage opening 21 of the wastewater, separated with reverse osmosis treatment through the reverse osmosis membrane apparatus 18 by switching the valve to “ON” or “OFF”. Wastewater is to be discharged from the drainage opening 21, when switching “ON” the drainage valve 23. Switching “ON” or “OFF” of the drainage valve 23 is controlled by the controller 25.

The control system is equipped with the controller 25 and the remote controller 26. The controller 25 runs the concerned control program on the main memory of the CPU, and controls the process of each processing components within the above-mentioned running water system.

The controller 25 regulates the pressure regulated by the low-pressure regulation unit 15, the pressure applied by the pressure pump 17, and the pressure and the water quantity regulated by the high-pressure regulation unit 19, to the value within the respective predefined range. In addition, the controller 25 regulates the temperature of water heated up by the heating unit 16 to the respective value range specified in advance. At this time, as the water temperature also depends on the water quantity running through the heating unit 16, in fact, it takes into consideration the value of flooding water quantity (40 cc/min-100 cc/min) from the high-pressure regulation unit 19 to control the temperature. In addition, the controller 25 controls the switching “ON” or “OFF” of the drainage valve 23 and solenoid valve 14, according to the control signal transmitted from the remote controller 26. Namely, it controls the simultaneous switching between the solenoid valve 14 and the drainage valve 23.

The remote controller 26 is provided separately from the cabinet 1 of the intestinal tract cleansing apparatus, and is placed in the position that is easy to use by the user of the intestinal tract cleansing apparatus (or toilet), for example. The user switches “ON” or OFF” of the solenoid valve 14 and the drainage valve 23, at the time of using the Intestinal tract cleansing apparatus. Accordingly, the remote controller 26 transmits the signal to the controller 25, to control the switching “ON” or “OFF” of the solenoid valve 14 and the drainage valve 23 by well-known wireless communication means. As such, the remote controller 26 and controller 25 are equipped with the transmitter-receiver for the wireless communications (not illustrated).

Next, it is explained to illustrate the example of processing at the time of using the intestinal tract cleansing apparatus, which adopts the interior configurations shown in FIG. 2. The user of the intestinal tract cleansing apparatus, at the time of using the pure water for cleansing (at the time of cleansing the intestinal tract), uses the remote controller 26 to switch “ON” the solenoid valve 14 and the drainage valve 23, after switching the switch 22 to the flooding opening 20 to direct the flooding of the pure water for cleansing.

By doing this, once the controller 25 switches “ON” the solenoid valve 14 and the drainage valve 23, the water from the faucet is to be introduced into the intestinal tract cleansing apparatus, and the first filter apparatus 11, the second filter apparatus 12, and the third filter apparatus 13, which comprise a set of the filter apparatus 10, filtrate the water introduced from the faucet in this order. The ultraviolet sterilizer 16′ kills the bacteria of the filtered water, and the heating unit 16 adjusts the water temperature to 37, for example. The pressure pump 17 adds the necessary pressure to reverse osmosis processing through the reverse osmosis membrane apparatus 18 onto the water introduced from heating unit 16. By doing this, the reverse osmosis membrane apparatus 18 carries out the reverse osmosis treatment, and generates the pure water for cleansing (and wastewater). The high-pressure regulation unit 19, sensors the water pressure of the pure water for cleansing, and regulates the pressure and quantity of the floodwater at the flooding opening 20 to the specified value. Consequently, it flushes out the pure water for cleansing through the flooding nozzle 24 of the flooding opening 20. The wastewater separated by the reverse osmosis membrane apparatus 18 is ejected from the drainage opening 21.

On the other hand, at the time of cleaning (at the time of the cleaning operation) the inside (of running water system) of the intestinal tract cleansing apparatus, the user of the intestinal tract cleansing apparatus uses the remote controller 26 to switch on the solenoid valve 14 and the drainage valve 23, after switching from the switch 22 to the drainage opening 21 to direct the flooding of the pure water for cleaning. By doing this, the drainage water from the reverse osmosis membrane apparatus 18, together with the pure water for cleansing, are ejected from the drainage opening 21 to clean up the inside of the running water system.

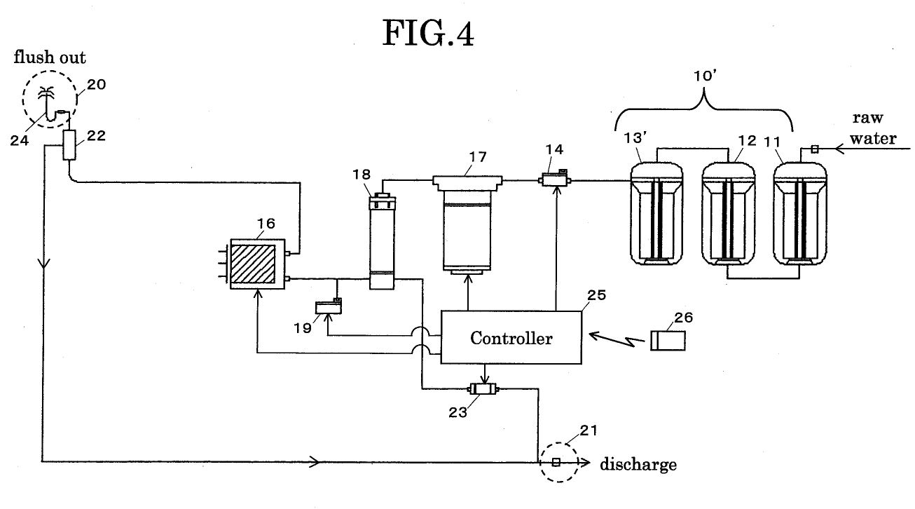

FIG. 4 shows an illustration of another configuration of the intestinal tract cleansing apparatus of the present invention. The intestinal tract cleansing apparatus of FIG. 4 is equipped primarily with the same configuration as the intestinal tract cleansing apparatus of FIG. 2, except that it differs from the illustration of FIG. 2 in respect of the followings. Namely, in the illustration in FIG. 4, the ultraviolet sterilizer 16′ is omitted. It is possible to eliminate the ultraviolet sterilizer 16′, since the reverse osmosis membrane apparatus 18 filters the bacteria and suchlike, and the pure water for cleansing does not contain bacteria. Moreover, it should not be a problem to add the ultraviolet sterilizer 16′ on the illustration of FIG. 4.

In addition, in the illustration of FIG. 4, the low-pressure regulation unit 15 is omitted. For example, it is possible to eliminate the low-pressure regulation unit 15 by regulating the pressure of the water from the faucet with the solenoid valve 14.

Moreover, in the illustration of FIG. 4, the heating unit 16 can be outfitted not at the direct front of the reverse osmosis membrane apparatus, but the switch 22 (a position nearer to the flooding opening 20 than the reverse osmosis membrane apparatus 18). Namely, the reverse osmosis membrane unit consisting of the reverse osmosis membrane apparatus 18 and the pressure pump 17 is to be provided not between the heating unit 16 and the flooding opening 20, but between the filter apparatus 10′ and the heating unit 16. By doing this, the heating unit 16 is nearer to the flooding opening 20 than to the reverse osmosis membrane unite, making it possible to regulate the water temperature of the pure water flooding from the flooding opening 20 more easily and accurately than the example of FIG. 2.

In addition, in the illustration of FIG. 4, the filter apparatus 10′ is to be provided, instead of the filter apparatus 10. The filter apparatus 10′ consists of the first filter apparatus 11, the second filter apparatus 12 and the third filter apparatus 13′. The third filter apparatus 13′, like the second filter apparatus 12, is equipped with an active carbon filter. With this, it can adsorb the chlorine, microorganism, organic matters and suchlike not filtered at the second filter apparatus 12, and prevent the clogging at the reverse osmosis membrane apparatus 18 provided at the back of the third filter apparatus 13′ within the concerned running water system. In addition, it is possible to remove effectively the chlorine, microorganism, organic matters and suchlike, which are the most serious problems of the current (convenient) water from the faucet, by providing the third filter 13′ equipped with the same active carbon filter separated from the second filter apparatus 12. Moreover, it is possible to replace either one of these separately. Because of this, it is possible to remove the chlorine, microorganism, organic matters and suchlike more effectively by separate replacement. Since the apparatus 12 and the apparatus 13′ equipped with an active carbon filter get clogged most easily when using raw water from the faucet.

The present invention may follow this form of execution as mentioned above. However, various modifications are possible within the scope of this tenor.

For example, the first filter apparatus 11 may be connected, for example, to tank instead of the faucet. In this case, the raw water is to be supplied from the tank, and the aperture of the raw water is to be supplied from the flooding opening of the tank. Consequently, it is possible to eliminate the need for connecting (construction work) the first filter apparatus 11 to the faucet. Moreover, it is possible to remove the necessity to prepare various joints to connect the faucet, which may have various forms and sizes depending on the country, region, makers and suchlike, with the first filter apparatus 11. The tank may be in the form, which can be installed on the surface, for example, of the cabinet 1. The capacity of the tank should be, for example, from tens to over several hundred of liters, and be able to handle a number of times of utilization. Consequently, it is possible to eliminate labor to supply the tank with water each time the apparatus is to be used.

As explained above, with the intestinal tract cleansing apparatus of the present invention, it is possible to obtain the pure water for cleansing by filtering the raw water from the faucet, for example, heat the pure water obtained for cleansing to body temperature, and introduce them into the intestinal tract at suitable pressure. Accordingly, it is possible to obtain the extremely pure water for intestinal tract cleansing, which is as clean as to satisfy the criterion of impurity concentration of potable water without having carcinogenic ozone and suchlike, bacteria and their residuals.

What is claimed is:

1. An intestinal tract cleansing apparatus that flushes output water for cleansing an intestinal tract from a flooding opening, the apparatus comprising:
a filter apparatus filtering water;
a heating unit heating the water; and
a reverse osmosis membrane unit treating the water with reverse osmosis,
wherein the water filtered with the filter apparatus is heated with the heating unit, and treated with reverse osmosis with the reverse osmosis membrane unit to output the water for cleansing an intestinal tract from the flooding opening.

2. The intestinal tract cleansing apparatus according to claim 1, further comprising:
a pressure regulation unit adjusting water pressure at the flooding opening.

3. The intestinal tract cleansing apparatus according to claim 2, further comprising:
a switching unit switching direction of the output of the water for cleansing the intestinal tract between the flooding opening and drainage opening provided separately with the flooding opening.

4. The intestinal tract cleansing apparatus according to claim 1, further comprising:
a switching unit switching direction of the output of the water for cleansing the intestinal tract between the flooding opening and drainage opening provided separately with the flooding opening.
